(19) United States
(12) Patent Application Publication
(10) Pub. No.: US 2024/0386535 A1
(43) Pub. Date: Nov. 21, 2024

(54) THREE-DIMENSIONAL IMAGE GENERATION METHOD AND THREE-DIMENSIONAL IMAGE GENERATION SYSTEM CAPABLE OF IMPROVING QUALITY OF A THREE-DIMENSIONAL IMAGE

(71) Applicant: QISIDA CORPORATION, Taoyuan City (TW)

(72) Inventors: Tsung-Hsi Lee, Taoyuan City (TW); Chuang-Wei Wu, Taoyuan City (TW); Min-Hsiung Huang, Taoyuan City (TW); Yen-Tsun Lin, Taoyuan City (TW)

(73) Assignee: QISIDA CORPORATION, tAOYUAN cITY (TW)

(21) Appl. No.: 18/662,956

(22) Filed: May 13, 2024

(30) Foreign Application Priority Data
May 17, 2023 (CN) 202310554055.6

(51) Int. Cl. G06T 5/70; G06T 5/50; G06T 7/246; G06T 15/00

(52) U.S. Cl. CPC G06T 5/70; G06T 5/50; G06T 7/246; G06T 15/00; G06T 2207/10028; G06T 2207/20212; G06T 2207/30036; G06T 2210/56

(57) ABSTRACT

A three-dimensional image generation method includes projecting a plurality of light patterns to an object to generate a plurality of groups of two-dimensional images, determining if the plurality of groups of two-dimensional images meet a predetermined rule, selecting j groups of two-dimensional images from the plurality of groups of two-dimensional images according to an image change amount of each group of two-dimensional image of the plurality of groups of two-dimensional images if the plurality of groups of two-dimensional images meet a predetermined rule, using the j groups of two-dimensional images to form j point clouds, and using the j point clouds to perform a splicing operation to generate a three-dimensional image. Determining if the plurality of groups of two-dimensional images meet the predetermined rule includes at least determining if the plurality of groups of two-dimensional images are corresponding to a same area.

### BACKGROUND OF THE INVENTION

### 1. Field of the Invention

The disclosure is related to a three-dimensional image generation method and a three-dimensional image generation system, and more particularly, a three-dimensional image generation method and a three-dimensional image generation system where some of two-dimensional images can be selected from a plurality of groups of two-dimensional images to generate a three-dimensional image with improved quality.

### 2. Description of the Prior Art

With the development of technology, more and more professionals begin to use optical auxiliary devices to improve the accuracy and convenience of related operations. For example, in dentistry, intraoral scanners can assist dentists in detecting oral cavities. An intraoral scanner can capture intraoral images and convert the images into digital data to assist professionals such as dentists and denture technicians for diagnosis, treatment and denture fabrication.

When an intraoral scanner is in use to obtain images of teeth, due to the limited space in the oral cavity, the user must continuously move the intraoral scanner to obtain a plurality of images. The plurality of images can be spliced to generate a more complete three-dimensional (3D) image.

In practice, the generated three-dimensional images are often deformed, resulting in poor image quality. According to analysis, the poor quality of the three-dimensional images is often caused by factors such as moving the scanner too fast or hand tremors. As a result, errors are accumulated after splicing multiple pieces of collected data, deteriorating the quality of the generated three-dimensional images. A solution is still in need to improve the quality of the generated three-dimensional images.

### SUMMARY OF THE INVENTION

An embodiment provides a three-dimensional image generation method. The method includes projecting a plurality of light patterns onto an object to generate a plurality of groups of two-dimensional images; capturing the plurality of groups of two-dimensional images;

determining if the plurality of groups of two-dimensional images meet predetermined rules; if the plurality of groups of two-dimensional images meet the predetermined rules, selecting j groups of two-dimensional images from the plurality of groups of two-dimensional images according to an image change amount of each group of two-dimensional images of the plurality of groups of two-dimensional images; using the j groups of two-dimensional images to generate j point clouds; and using the j point clouds to perform a splicing operation to generate a three-dimensional image. In the method, j is an integer larger than 1, and determining if the plurality of groups of two-dimensional images meet the predetermined rules comprises determining if the plurality of groups of two-dimensional images are corresponding to a same area.

Another embodiment provides a three-dimensional image generation method. The method includes projecting a plurality of light patterns onto an object to generate a plurality of groups of two-dimensional images; capturing the plurality of groups of two-dimensional images; using the plurality of groups of two-dimensional images to generate a plurality of point clouds; determining if the plurality of groups of two-dimensional images meet predetermined rules; if the plurality of groups of two-dimensional images meet the predetermined rules, selecting j groups of two-dimensional images from the plurality of groups of two-dimensional images according to an image change amount of each group of two-dimensional images of the plurality of groups of two-dimensional images; selecting i point clouds from j point clouds according to depth-of-field information of each point cloud of the j point clouds; and using the i point clouds to perform a splicing operation to generate a three-dimensional image. The j point clouds are corresponding to the j groups of two-dimensional images, the plurality of point clouds comprise the j point clouds, i and j are integers larger than 1, and determining if the plurality of groups of two-dimensional images meet the predetermined rules comprises determining if the plurality of groups of two-dimensional images are corresponding to a same area.

Another embodiment provides a three-dimensional image generation system. The system includes a projector, a camera and a processor. The projector is used to project a plurality of light patterns onto an object to generate a plurality of groups of two-dimensional images. The camera is used to capture the plurality of groups of two-dimensional images. The processor is coupled to the projector and the camera, and used to generate the plurality of light patterns, receive the plurality of groups of two-dimensional images, determine if the plurality of groups of two-dimensional images meet predetermined rules, select x groups of two-dimensional images from the plurality of groups of two-dimensional images according to an image change amount of each group of two-dimensional images of the plurality of groups of two-dimensional images if the plurality of groups of two-dimensional images meet the predetermined rules, generate a plurality of point clouds according to a plurality of groups of two-dimensional images of the x groups of two-dimensional images, and use the plurality of point clouds to perform a splicing operation to generate a three-dimensional image. In the system, x is an integer larger than 1, and whether the plurality of groups of two-dimensional images meet the predetermined rules is determined by at least whether the plurality of groups of two-dimensional images are corresponding to a same area.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 illustrates a three-dimensional generation system according to an embodiment.

FIG. 2 illustrates a group of light patterns according to an embodiment.

FIG. 3 is a flowchart of a three-dimensional image generation method for the three-dimensional image generation system in FIG. 1 according to an embodiment.

FIG. 4 illustrates image gradient change amounts of a two-dimensional image according to an embodiment.

FIG. 5 is a flowchart of a three-dimensional image generation method for the three-dimensional image generation system in FIG. 1 according to another embodiment.

FIG. 6 is a flowchart of a three-dimensional image generation method for the three-dimensional image generation system in FIG. 1 according to another embodiment.

FIG. 7 is a flowchart for performing compensation to improve the three-dimensional image of FIG. 1.

FIG. 8 illustrates the first group of two-dimensional images and the second group of two-dimensional images mentioned in FIG. 7.

FIG. 9 illustrates a comparison of an image of the first group of two-dimensional images in FIG. 7 and an image of the second group of two-dimensional images in FIG. 7.

### DETAILED DESCRIPTION

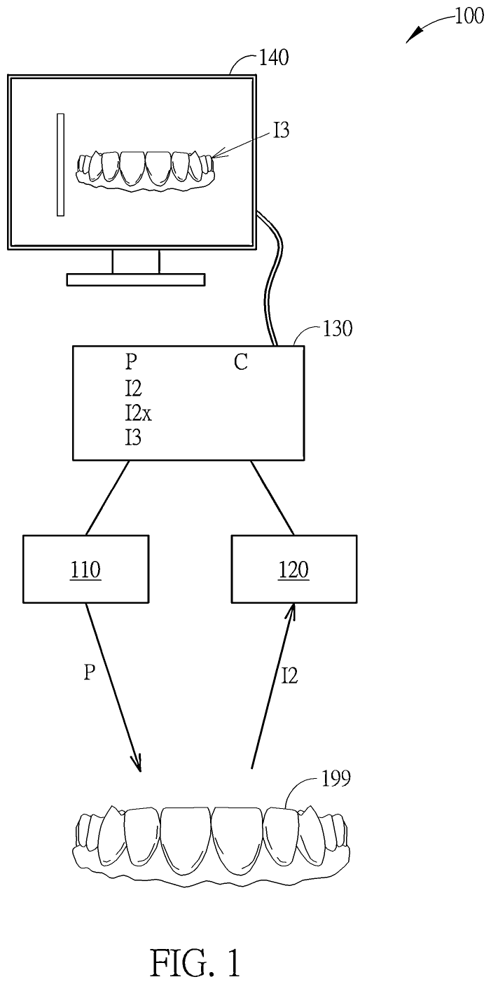

FIG. 1 illustrates a three-dimensional generation system 100 according to an embodiment. The three-dimensional generation system 100 can include a projector 110, a camera 120, a processor 130 and a display 140. The projector 110 can project a plurality of light patterns P onto an object 199 to generate a plurality of groups of two-dimensional images I2. The camera 120 can capture the plurality of groups of two-dimensional images I2. The projector 110 and the camera 120 can be disposed in a movable device, such as a handheld portion of an intraoral scanner.

In the text, a group of two-dimensional images can be used to form a point cloud, and each point cloud can be formed with a plurality of points. For example, a group of two-dimensional images can include (but not be limited to) 8 two-dimensional images, and 8 two-dimensional images can be used to form one point cloud. More details and examples are described below.

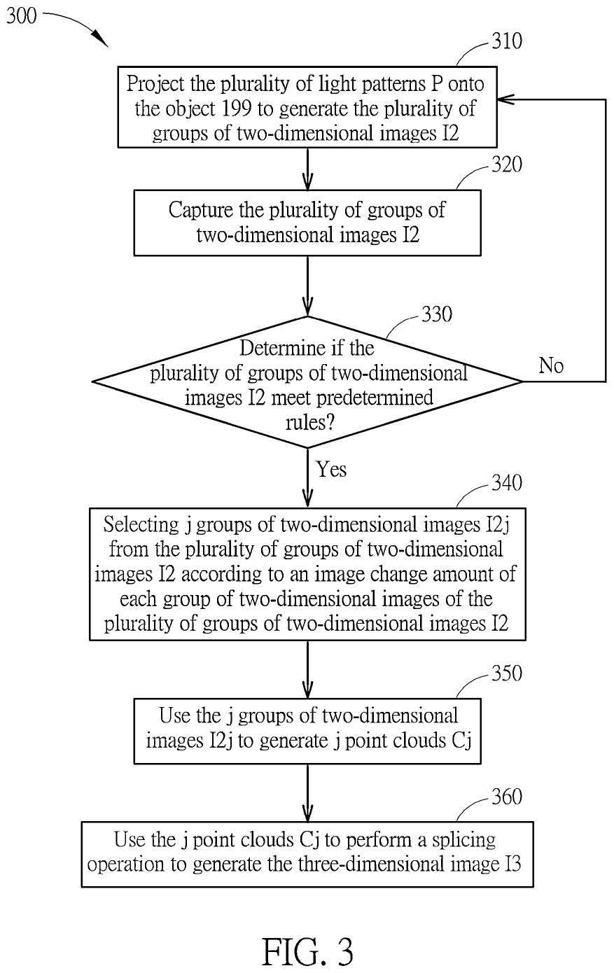

The processor 130 can be coupled to the projector 110 and the camera 120. The processor 130 can provide the light patterns P, receive the plurality of groups of two-dimensional images I2, and determine whether the plurality of groups of two-dimensional images I2 meet predetermined rules. If the plurality of groups of two-dimensional images I2 meet predetermined rules, the processor 130 can select x groups of two-dimensional images I2x from the plurality of groups of two-dimensional images I2 according to an image change amount of each group of two-dimensional images of the plurality of groups of two-dimensional images I2. The processor 130 can generate x′ point clouds C according to x′ groups of two-dimensional images of the x groups of two-dimensional images I2x, and use the x′ point clouds C to perform a splicing operation to generate a three-dimensional image I3. The x groups of two-dimensional images I2x can be a subset of the plurality of groups of two-dimensional images I2, or the x groups of two-dimensional images I2x can include all of the plurality of groups of two-dimensional images I2. The parameter x can be an integer larger than 1, and the parameter x can be greater than or equal to the parameter x′. The processor 130 can determine whether the plurality of groups of two-dimensional images I2 meet the predetermined rules by at least determining whether the plurality of groups of two-dimensional images I2 are corresponding to the same area, and this can be corresponding to Step 330 in FIG. 3. More details are described below.

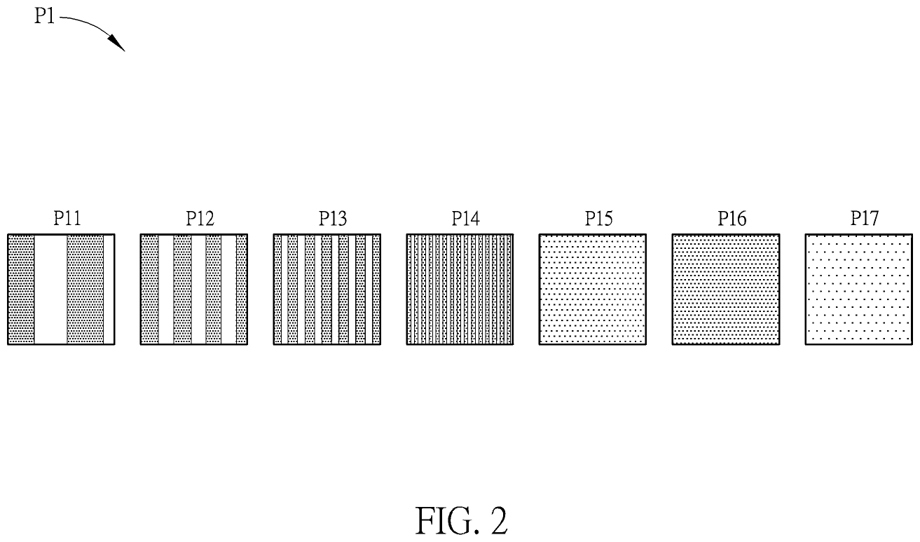

FIG. 2 illustrates a group of light patterns P1 according to an embodiment. The light patterns P1 can be corresponding to the light patterns P mentioned in FIG. 1. FIG. 2 is an example, embodiments are not limited thereto, and other proper light patterns can be used. The light patterns P1 can include light patterns P11 to P17. The light patterns P11 to P14 can be stripe patterns with different pitches and stripe widths. For example, the light patterns P11 to P14 may include at least one of a gray code pattern and a line shift pattern. In another embodiment, the light patterns P11 to P14 may be light patterns with different image gradients. The light patterns P15 to P17 may be light patterns of different colors, such as red, green and blue (often abbreviated as R, G and B) light patterns. By projecting the light patterns P1 to the object 199 and then receiving the reflected images, a plurality of groups of two-dimensional images of the object 199 can be generated accordingly, thereby generating a point cloud according to the two-dimensional images. Then, point clouds can be spliced to generate a three-dimensional image accordingly.

FIG. 3 is a flowchart of a three-dimensional image generation method 300 for the three-dimensional image generation system 100 in FIG. 1 according to an embodiment. The three-dimensional image generation method 300 may include the following steps.

Step 310: project the plurality of light patterns P onto the object 199 to generate the plurality of groups of two-dimensional images I2;

Step 320: capture the plurality of groups of two-dimensional images I2;

Step 330: determine if the plurality of groups of two-dimensional images I2 meet predetermined rules? If so, enter Step 340; otherwise, enter Step 310;

Step 340: selecting j groups of two-dimensional images (expressed as I2j) from the plurality of groups of two-dimensional images I2 according to an image change amount of each group of two-dimensional images of the plurality of groups of two-dimensional images I2;

Step 350: use the j groups of two-dimensional images I2j to generate j point clouds (expressed as Cj); and

Step 360: use the j point clouds Cj to perform a splicing operation to generate the three-dimensional image I3.

The parameter j mentioned above may be an integer larger than 1. In Step 310, the plurality of light patterns P can include (but not be limited to) the light patterns in FIG. 2. In Step 320, each group of two-dimensional images of the plurality of groups of two-dimensional images I2 can be used to generate one three-dimensional point cloud.

Step 330 may include preliminary determination, and Step 330 may include at least determining whether the plurality of groups of two-dimensional images I2 are corresponding to a same area. If the plurality of groups of two-dimensional images I2 are not corresponding to a same area, it means that the speed of moving the projector 110 (for example, the handheld part of the intraoral scanner) may be too high. At this time, the subsequent Step 340 to Step 360 may be optionally omitted to avoid producing low-quality three-dimensional images.

In addition, Step 330 can further include determining if the plurality of groups of two-dimensional images I2 have reached a predetermined number of groups, where the predetermined number can be greater than the parameter j in Step 340. The parameter j in FIG. 3 can be equal to or smaller than the parameter x in FIG. 1.

In Step 340, at least one group of two-dimensional images with a larger image change amount can be abandoned to reduce unwanted ripples, noises and deformations in the three-dimensional image generated accordingly. As a result, the quality of the three-dimensional image is improved. More details related to Step 340 are described below.

In Step 350 and Step 360, the j groups of two-dimensional images I2j with smaller image change amounts can be selected to form the j point clouds Cj, and the j point clouds Cj can be spliced accordingly to generate the three-dimensional image I3 with higher quality. In Step 360, the splicing operation can include a three-dimensional registration operation, such as an iterative closest point (ICP) operation. After performing Step 360, scanning can be stopped and the generated three-dimensional image can be used for observation or other post-processing.

In another embodiment, the operation of generating the point clouds described in Step 350 may optionally be performed before Step 330. For example, the plurality of groups of two-dimensional images I2 can be converted into a plurality of point clouds. Then, after Step 340, the j point clouds Cj corresponding to the j groups of two-dimensional images I2j can be selected from the plurality of point clouds for splicing.

According to embodiments, in Step 340, at least one of the following operation OP1, operation OP2 and operation OP3 may be performed.

### The Operation OP1:

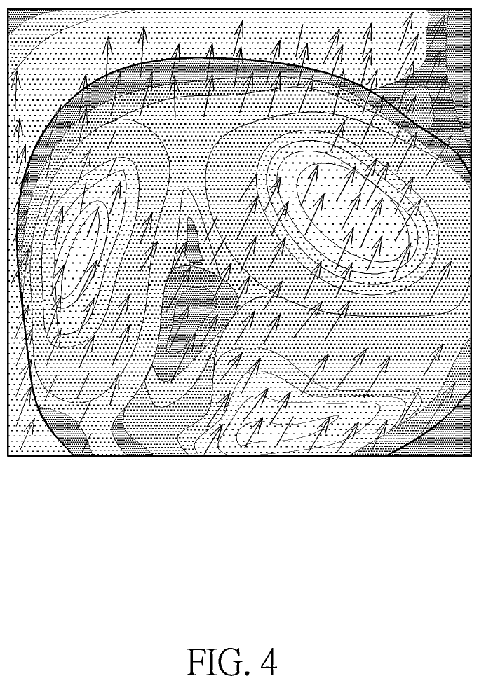

In Step 340, if an image gradient change amount of a group of two-dimensional images of the plurality of groups of two-dimensional images I2 exceeds a threshold, the group of two-dimensional images can be abandoned for selecting the j groups of two-dimensional images I2j from the plurality of groups of two-dimensional images I2. FIG. 4 illustrates image gradient change amounts of a two-dimensional image according to an embodiment. An arrow in FIG. 4 can be a direction of an image gradient change in one area. For example, a single two-dimensional image can be divided into 20×20 small areas (i.e. 400 small areas), and the changes of the image gradient (e.g. grayscale gradient) of each small area can be observed and calculated by summing or performing a predetermined calculation operation to generate the image gradient change amount of the single two-dimensional image.

If an image gradient change amount of a single two-dimensional image is greater than a threshold, it means a problem of moving a scanner too fast or hand tremor may occur when operating the projector 110 and camera 120.

### The Operation OP2:

In Step 340, if an optical flow change amount of a group of two-dimensional images of the plurality of groups of two-dimensional images I2 exceeds a threshold, the group of two-dimensional images can be abandoned for selecting the j groups of two-dimensional images I2j from the plurality of groups of two-dimensional images I2. An optical flow can be a pattern of an apparent motion of an object, surface and edge in a visual scene caused by a relative motion between an observer and an observed object. For example, if in a group of two-dimensional images, a movement of an observed pattern between one two-dimensional image and another two-dimensional image is too large, it means a problem of moving a scanner too fast or hand tremor may occur when operating the projector 110 and camera 120.

### The Operation OP3:

In Step 340, if a feature point movement of a group of two-dimensional images of the plurality of groups of two-dimensional images I2 exceeds a threshold, the group of two-dimensional images can be abandoned for selecting the j groups of two-dimensional images I2j from the plurality of groups of two-dimensional images I2. For example, when an intraoral scanner is used to scan teeth, if a specific spot on a specific tooth moves too much from one two-dimensional image to another two-dimensional image in a group of two-dimensional images, it means a problem of moving a scanner too fast or hand tremor may occur when operating the projector 110 and camera 120.

In Step 340, by performing at least one of the operations OP1, OP2, and OP3, at least one group of two-dimensional images with an excessive image change amount can be abandoned to avoid generating related point clouds, thereby avoiding the degradation of the quality of the subsequently generated three-dimensional image I3. In other words, the quality of the three-dimensional image I3 is improved.

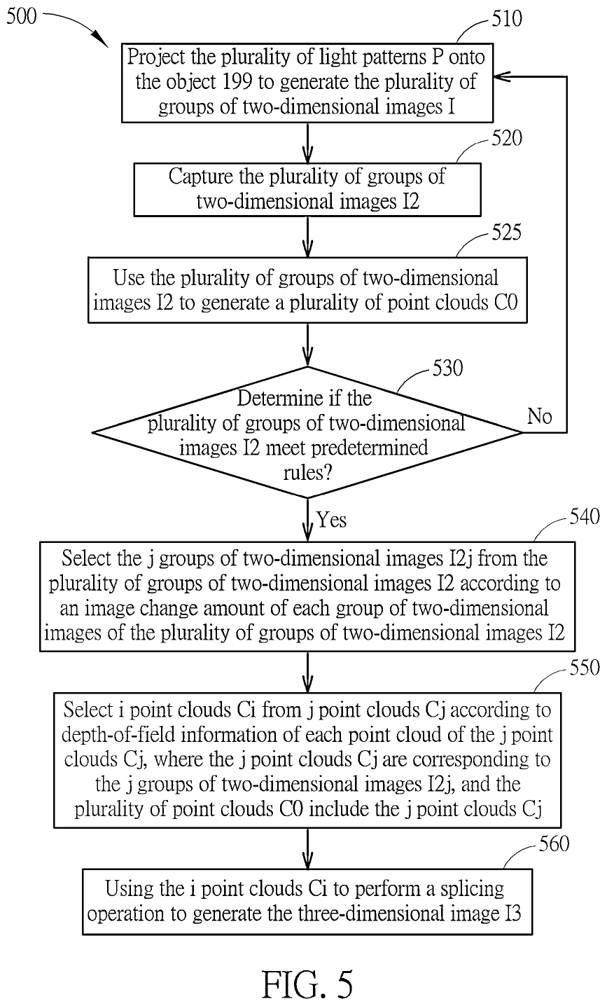

FIG. 5 is a flowchart of a three-dimensional image generation method 500 for the three-dimensional image generation system 100 in FIG. 1 according to another embodiment. The three-dimensional image generation method 500 may include the following steps.

Step 510: project the plurality of light patterns P onto the object 199 to generate the plurality of groups of two-dimensional images I2;

Step 520: capture the plurality of groups of two-dimensional images I2;

Step 525: use the plurality of groups of two-dimensional images I2 to generate a plurality of point clouds (expressed as CO);

Step 530: determine if the plurality of groups of two-dimensional images I2 meet predetermined rules? If so, enter Step 540; otherwise, enter Step 510;

Step 540: select the j groups of two-dimensional images I2j from the plurality of groups of two-dimensional images I2 according to an image change amount of each group of two-dimensional images of the plurality of groups of two-dimensional images I2;

Step 550: select i point clouds (expressed as Ci) from j point clouds (expressed as Cj) according to depth-of-field information of each point cloud of the j point clouds Cj, where the j point clouds Cj are corresponding to the j groups of two-dimensional images I2j, and the plurality of point clouds CO include the j point clouds Cj; and

Step 560: using the i point clouds Ci to perform a splicing operation to generate the three-dimensional image I3.

In FIG. 5, the parameters j and i can be integers, and j≥i>1. Step 510 and Step 530 can be similar to Step 310 and Step 320. Like Step 330 of FIG. 3, in Step 530, determining if the plurality of groups of two-dimensional images I2 meet the predetermined rules can include at least determining if the plurality of groups of two-dimensional images I2 are corresponding to a same area. If the result of Step 530 is “no”, it means the speed of moving the projector 110 (e.g. a hand-held part of an intraoral scanner) may be too high, and subsequent Step 540 to Step 560 may be optionally omitted to avoid generating the three-dimensional image I3 with low quality. In addition, Step 530 may further include determining if the plurality of groups of two-dimensional images I2 have reached a predetermined number of groups, and the predetermined number may be greater than the parameter j in Step 540. The parameter j in FIG. 5 can be equal to or smaller than the parameter x in FIG. 1.

In Step 525, the plurality of groups of two-dimensional images I2 can all be converted to generate the plurality of point clouds CO, and a part of the plurality of point clouds CO (i.e. the i point clouds Ci) can be selected in Step 550.

Step 540 can be similar to Step 340 of FIG. 3. In Step 540, at least one of the abovementioned operations OP1, OP2 and OP3 can be performed to select the j groups of two-dimensional images I2j from the plurality of groups of two-dimensional images I2 and abandon at least one group of two-dimensional images with an excessive image change amount. Hence, point clouds related to the abandoned two-dimensional images are not generated, thereby avoiding the degradation of the quality of three-dimensional images generated subsequently.

In FIG. 5, Step 525 can precede Step 550, so that in Step 550, each point cloud can be determined to be retained or abandoned according to depth-of-field information of each point cloud.

Step 550 can include at least one of the following operations OPA and OPB.

### The Operation OPA:

If a percentage of depth-of-field information of a point cloud of the j point clouds Cj higher than an upper limit is greater than a first threshold, and/or if a percentage of the depth-of-field information of the point cloud of the j point clouds Cj lower than a lower limit is greater than a second threshold, the point cloud can be abandoned, thereby selecting the i point clouds Ci. For example, according to depth-of-field information of a point cloud, if the percentage of the area of the object 199 too close to the projector 110 and the camera 120 (e.g. a front end of a hand-held portion of an intraoral scanner) is excessive, the point cloud may be abandoned. In another example, according to depth-of-field information of a point cloud, if the percentage of the area of the object 199 too far away from the projector 110 and the camera 120 is excessive, the point cloud may be abandoned.

### The Operation OPA:

A queue can be generated according to depth-of-field information of the j point clouds Cj, and the i point clouds Ci can be selected from the j point clouds Cj according to the queue. For example, the j point clouds Cj can be arranged into a first point cloud to a jth point cloud according to the depth-of-field information, and point clouds with deeper and shallower depth-of-field information can be abandoned to select the i point clouds Ci.

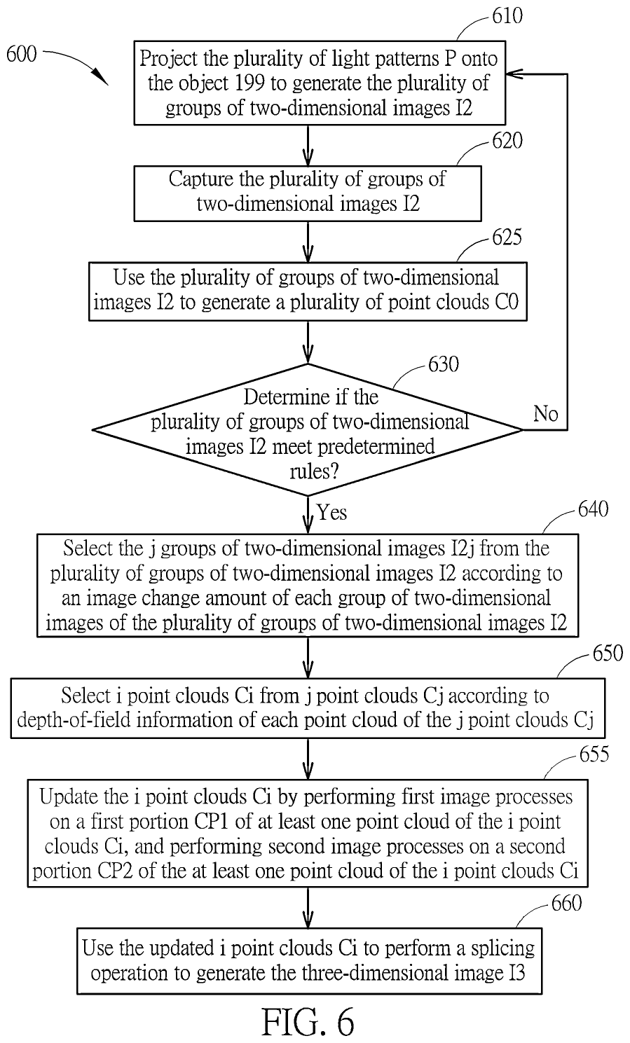

FIG. 6 is a flowchart of a three-dimensional image generation method 600 for the three-dimensional image generation system 100 in FIG. 1 according to another embodiment. The three-dimensional image generation method 600 may include the following steps.

Step 610: project the plurality of light patterns P onto the object 199 to generate the plurality of groups of two-dimensional images I2;

Step 620: capture the plurality of groups of two-dimensional images I2;

Step 625: use the plurality of groups of two-dimensional images I2 to generate a plurality of point clouds (expressed as CO);

Step 630: determine if the plurality of groups of two-dimensional images I2 meet predetermined rules? If so, enter Step 640; otherwise, enter Step 610;

Step 640: select the j groups of two-dimensional images I2j from the plurality of groups of two-dimensional images I2 according to an image change amount of each group of two-dimensional images of the plurality of groups of two-dimensional images I2;

Step 650: select i point clouds (expressed as Ci) from j point clouds (expressed as Cj) according to depth-of-field information of each point cloud of the j point clouds Cj, where the j point clouds Cj are corresponding to the j groups of two-dimensional images I2j, and the plurality of point clouds CO include the j point clouds Cj;

Step 655: update the i point clouds Ci by performing first image processes on a first portion (expressed as CP1) of at least one point cloud of the i point clouds Ci, and performing second image processes on a second portion (expressed as CP2) of the at least one point cloud of the i point clouds Ci; and

Step 660: use the updated i point clouds Ci to perform a splicing operation to generate the three-dimensional image I3.

Step 610 to Step 650 and Step 660 in FIG. 6 can be similar to Step 510 to Step 550 and Step 560, so it is not repeatedly described. Compared with FIG. 5, FIG. 6 can further include Step 655. In Step 655, in the processed point clouds, image quality of the first portion CP1 can be inferior than image quality of the second portion CP2. The number of the first image processes can be greater than the number of the second image processes. A parameter of the first image processes can be greater than or smaller than a parameter of the second image processes, and it depends on requirements of image and the purpose of the processing parameter.

For example, in Step 655, when performing the splicing operation, more smoothing processes and/or a larger smoothing parameter can be applied to blurry parts of the point clouds with a deeper or shallower depth-of-field (i.e. the first portion CP1). Less smoothing processes and/or a smaller smoothing parameter can be applied to clearer parts of the point clouds with a moderate depth-of-field (i.e. the second portion CP2). In another case, for a purpose of generating an accurate image, a processing parameter for the first portion CP1 can be smaller than a processing parameter for the second portion CP2. In FIG. 6, by performing Step 655 to perform different processes for different portions of the point clouds, the quality of the three-dimensional image I3 is further improved.

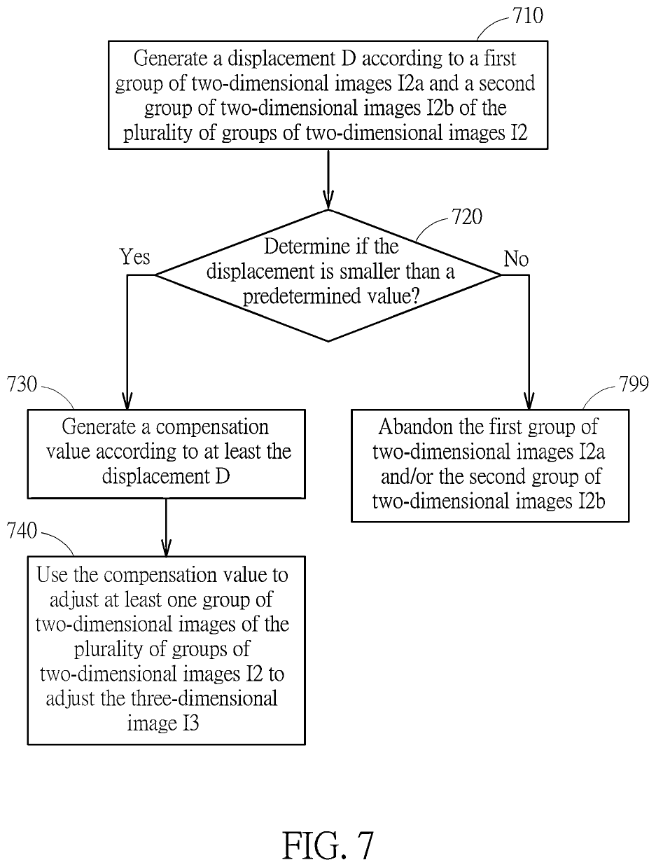

FIG. 7 is a flowchart for performing compensation to improve the three-dimensional image I3 of FIG. 1. When performing the flows in FIG. 3, FIG. 5 and FIG. 6, the flow in FIG. 7 can be optionally performed for compensation to further improve the three-dimensional image I3. The flow in FIG. 7 can include the following steps.

Step 710: generate a displacement (expressed as D) according to a first group of two-dimensional images (expressed as I2a) and a second group of two-dimensional images (expressed as I2b) of the plurality of groups of two-dimensional images I2;

Step 720: determine if the displacement is smaller than a predetermined value? if so, enter Step 730; otherwise, enter Step 799;

Step 730: generate a compensation value according to at least the displacement D;

Step 740: use the compensation value to adjust at least one group of two-dimensional images of the plurality of groups of two-dimensional images I2 to adjust the three-dimensional image I3; and

Step 799: abandon the first group of two-dimensional images I2a and/or the second group of two-dimensional images I2b.

In Step 710, pre-processing can be performed on the plurality of groups of two-dimensional images I2. For example, the pre-processing may include smoothing technique(s) such as Gaussian blur, median filtering and/or mean filtering for reducing noises. The pre-processing may be performed to normalize brightness. In addition, image features (e.g. contours, phases, brightness, etc.) of the first group of two-dimensional images I2a and the second group of two-dimensional images I2b can be captured to generate the displacement D accordingly.

In Step 720, if the displacement D is excessive, it means that it is no longer possible to compensate and improve the three-dimensional image I3. Therefore, Step 799 can be entered to discard the unusable two-dimensional images. Then, the object 199 can be scanned to collect new two-dimensional images, or the flow can be ended.

In Step 720, if the displacement D is not excessive, the flow can enter Step 730 to generate the compensation value, and related principle is described below. In Step 730 and Step 740, a rotation transformation matrix (RT matrix) used in three-dimensional registration can be adjusted according to the compensation value. By detecting and compensating the motion of the object, the result of splicing three-dimensional point clouds is improved to improve the quality of the three-dimensional image I3.

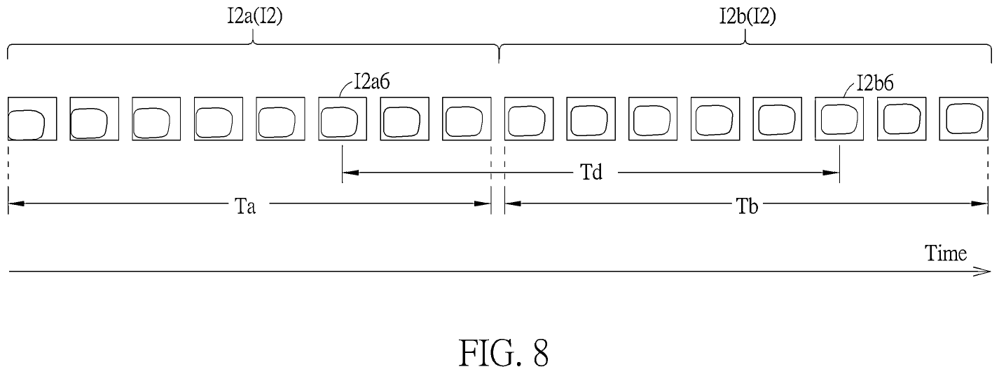

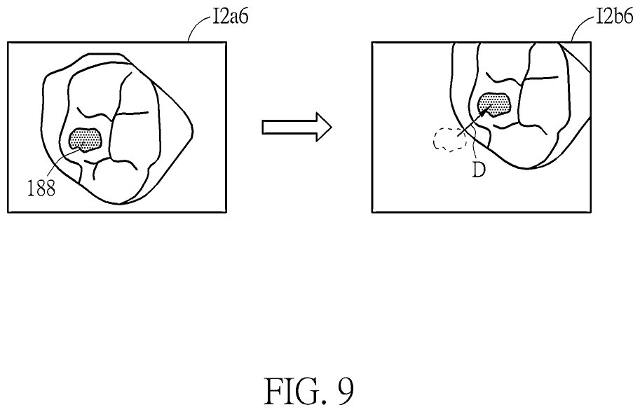

FIG. 8 illustrates the first group of two-dimensional images I2a and the second group of two-dimensional images I2b mentioned in FIG. 7. FIG. 9 illustrates a comparison of an image I2a6 of the first group of two-dimensional images I2a in FIG. 7 and an image I2b6 of the second group of two-dimensional images I2b in FIG. 7. FIG. 8 and FIG. 9 are examples for describing the process, and embodiments are not limited thereto.

As shown in FIG. 8, the first group of two-dimensional images I2a and the second group of two-dimensional images I2b may each include eight two-dimensional images. The first group of two-dimensional images I2a may be corresponding to a time period Ta, and the second group of two-dimensional images I2b may be corresponding to a time period Tb, and the time period Ta may precede the time period Tb. The image I2a6 may be the sixth image of the first group of two-dimensional images I2a, and the image I2b6 may be the sixth image of the second group of two-dimensional images I2b. As shown in FIG. 9, from the image I2a6 to the image I2b6, a feature point 188 of the object 199 (e.g. a spot on a tooth) can move by a displacement D, where the displacement D can be expressed in two-dimensional coordinates.

There may be a time difference (expressed as Td) between the first group of two-dimensional images I2a and the second group of two-dimensional images I2b. For example, there may be the time difference Td between the image I2a6 and the image I2b6. Hence, the feature point 188 can move by the displacement amount D within the time difference Td, thereby generating a speed of the movement of the feature point 188. Therefore, a compensation value can be generated according to the displacement D and the time difference Td to compensate and correct the rotation transformation matrix (RT matrix) to improve the quality of the generated three-dimensional image I3.

In this example, between the sixth image of the first group of two-dimensional images I2a (e.g. I2a6) and the sixth image of the second group of two-dimensional images I2b (e.g. I2b6), after the time difference Td and the displacement amount D of the feature point 188 are generated, the displacements and time differences between a plurality of images of the first group of two-dimensional images I2a and a plurality of images of the second group of two-dimensional images I2b can be calculated accordingly and be used for compensating each two-dimensional image to adjust results of three-dimensional registration.

After the displacement D is generated, a post-processing parameter can be generated according to the displacement D. The post-processing parameter can be used to perform a de-noising process and/or a smoothening process on the three-dimensional images that have not been spliced to further improve the quality of the three-dimensional image I3 generated by the splicing operation.

In summary, by selecting two-dimensional images with smaller image change amounts to be spliced to generate the three-dimensional image I3, noise in the three-dimensional image I3 (e.g. unwanted ripples and rough bulges) is reduced. In addition, by detecting the displacement between two groups of two-dimensional images, motion compensation and de-noising processing can be performed accordingly to further improve the three-dimensional image I3. As a result, problems in the field of three-dimensional imaging are effectively reduced.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A three-dimensional image generation method comprising:
projecting a plurality of light patterns onto an object to generate a plurality of groups of two-dimensional images;
capturing the plurality of groups of two-dimensional images;
determining if the plurality of groups of two-dimensional images meet predetermined rules;
if the plurality of groups of two-dimensional images meet the predetermined rules, selecting j groups of two-dimensional images from the plurality of groups of two-dimensional images according to an image change amount of each group of two-dimensional images of the plurality of groups of two-dimensional images;
using the j groups of two-dimensional images to generate j point clouds; and
using the j point clouds to perform a splicing operation to generate a three-dimensional image;
wherein j is an integer larger than 1, and determining if the plurality of groups of two-dimensional images meet the predetermined rules comprises determining if the plurality of groups of two-dimensional images are corresponding to a same area.

2. The method of claim 1, wherein determining if the plurality of groups of two-dimensional images meet the predetermined rules further comprises determining if the plurality of groups of two-dimensional images have reached a predetermined number of groups, the predetermined number being greater than j.

3. The method of claim 1, wherein selecting the j groups of two-dimensional images from the plurality of groups of two-dimensional images according to the image change amount of each group of two-dimensional images of the plurality of groups of two-dimensional images comprises:
if an optical flow change amount of a group of two-dimensional images of the plurality of groups of two-dimensional images exceeds a threshold, abandoning the group of two-dimensional images.

4. The method of claim 1, wherein selecting the j groups of two-dimensional images from the plurality of groups of two-dimensional images according to the image change amount of each group of two-dimensional images of the plurality of groups of two-dimensional images comprises:
if a feature point movement of a group of two-dimensional images of the plurality of groups of two-dimensional images exceeds a threshold, abandoning the group of two-dimensional images.

5. The method of claim 1, wherein selecting the j groups of two-dimensional images from the plurality of groups of two-dimensional images according to the image change amount of each group of two-dimensional images of the plurality of groups of two-dimensional images comprises:
if an image gradient change amount of a group of two-dimensional images of the plurality of groups of two-dimensional images exceeds a threshold, abandoning the group of two-dimensional images.

6. The method of claim 1, further comprising:
generating a displacement according to a first group of two-dimensional images and a second group of two-dimensional images of the plurality of groups of two-dimensional images;
if the displacement is smaller than a predetermined value, generating a compensation value according to at least the displacement; and
using the compensation value to adjust at least one group of two-dimensional images of the plurality of groups of two-dimensional images to adjust the three-dimensional image.

7. The method of claim 6, further comprising:
generating a time difference according to the first group of two-dimensional images and the second group of two-dimensional images;
wherein if the displacement is smaller than the predetermined value, the compensation value is generated according to the displacement and the time difference.

8. The method of claim 6, further comprising:
generating a post-processing parameter according to the displacement; and
using the post-processing parameter to perform a de-noising process and/or a smoothening process on the three-dimensional image.

9. A three-dimensional image generation method comprising:
projecting a plurality of light patterns onto an object to generate a plurality of groups of two-dimensional images;
capturing the plurality of groups of two-dimensional images;
using the plurality of groups of two-dimensional images to generate a plurality of point clouds;
determining if the plurality of groups of two-dimensional images meet predetermined rules;
if the plurality of groups of two-dimensional images meet the predetermined rules, selecting j groups of two-dimensional images from the plurality of groups of two-dimensional images according to an image change amount of each group of two-dimensional images of the plurality of groups of two-dimensional images;
selecting i point clouds from j point clouds according to depth-of-field information of each point cloud of the j point clouds; and
using the i point clouds to perform a splicing operation to generate a three-dimensional image;
wherein the j point clouds are corresponding to the j groups of two-dimensional images, the plurality of point clouds comprise the j point clouds, i and j are integers, j≥i>1, and determining if the plurality of groups of two-dimensional images meet the predetermined rules comprises determining if the plurality of groups of two-dimensional images are corresponding to a same area.

10. The method of claim 9, wherein selecting the i point clouds from the j point clouds according to the depth-of-field information of each point cloud of the j point clouds comprises:
if a percentage of depth-of-field information of a point cloud of the j point clouds higher than an upper limit is greater than a first threshold, and/or if a percentage of the depth-of-field information of the point cloud of the j point clouds lower than a lower limit is greater than a second threshold, abandoning the point cloud.

11. The method of claim 9, wherein selecting the i point clouds from the j point clouds according to the depth-of-field information of each point cloud of the j point clouds, comprises:
generating a queue according to depth-of-field information of the j point clouds; and
selecting the i point clouds from the j point clouds according to the queue.

12. The method of claim 9, further comprising:
updating the i point clouds to generate updated i point clouds by performing first image processes on a first portion of at least one point cloud of the i point clouds, and performing second image processes on a second portion of the at least one point cloud of the i point clouds;
wherein image quality of the first portion is inferior than image quality of the second portion;
number of the first image processes is greater than number of the second image processes, and/or a parameter of the first image processes is greater than a parameter of the second image processes; and
the splicing operation is performed using the updated i point clouds.

13. The method of claim 9, wherein determining if the plurality of groups of two-dimensional images meet the predetermined rules further comprises determining if the plurality of groups of two-dimensional images have reached a predetermined number of groups, the predetermined number being greater than j.

14. The method of claim 9, wherein selecting the j groups of two-dimensional images from the plurality of groups of two-dimensional images according to the image change amount of each group of two-dimensional images of the plurality of groups of two-dimensional images comprises:
if an optical flow change amount of a group of two-dimensional images of the plurality of groups of two-dimensional images exceeds a threshold, abandoning the group of two-dimensional images.

15. The method of claim 9, wherein selecting the j groups of two-dimensional images from the plurality of groups of two-dimensional images according to the image change amount of each group of two-dimensional images of the plurality of groups of two-dimensional images comprises:
if a feature point movement of a group of two-dimensional images of the plurality of groups of two-dimensional images exceeds a threshold, abandoning the group of two-dimensional images.

16. The method of claim 9, wherein selecting the j groups of two-dimensional images from the plurality of groups of two-dimensional images according to the image change amount of each group of two-dimensional images of the plurality of groups of two-dimensional images comprises:
if an image gradient change amount of a group of two-dimensional images of the plurality of groups of two-dimensional images exceeds a threshold, abandoning the group of two-dimensional images.

17. The method of claim 9, further comprising:
generating a displacement according to a first group of two-dimensional images and a second group of two-dimensional images of the plurality of groups of two-dimensional images;
if the displacement is smaller than a predetermined value, generating a compensation value according to at least the displacement; and
using the compensation value to adjust at least one group of two-dimensional images of the plurality of groups of two-dimensional images to adjust the three-dimensional image.

18. The method of claim 17, further comprising:
generating a time difference according to the first group of two-dimensional images and the second group of two-dimensional images;
wherein if the displacement is smaller than the predetermined value, the compensation value is generated according to the displacement and the time difference.

19. The method of claim 17, further comprising:
generating a post-processing parameter according to the displacement; and
using the post-processing parameter to perform a de-noising process and/or a smoothening process on the three-dimensional image.

20. A three-dimensional image generation system, comprising:
a projector configured to project a plurality of light patterns onto an object to generate a plurality of groups of two-dimensional images;
a camera configured to capture the plurality of groups of two-dimensional images; and
a processor coupled to the projector and the camera, and configured to generate the plurality of light patterns, receive the plurality of groups of two-dimensional images, determine if the plurality of groups of two-dimensional images meet predetermined rules, select x groups of two-dimensional images from the plurality of groups of two-dimensional images according to an image change amount of each group of two-dimensional images of the plurality of groups of two-dimensional images if the plurality of groups of two-dimensional images meet the predetermined rules, generate a plurality of point clouds according to a plurality of groups of two-dimensional images of the x groups of two-dimensional images, and use the plurality of point clouds to perform a splicing operation to generate a three-dimensional image;
wherein x is an integer larger than 1, and whether the plurality of groups of two-dimensional images meet the predetermined rules is determined by at least whether the plurality of groups of two-dimensional images are corresponding to a same area.
